(19) United States
(12) Patent Application Publication
(10) Pub. No.: US 2025/0043245 A1
(43) Pub. Date: Feb. 6, 2025

(54) METHOD OF PRODUCING AN IMMUNE CELL THAT RESPONDS TO A CANCER-ASSOCIATED ANTIGEN

(71) Applicants: Koji Tanabe, Palo Alto, CA (US); I Peace, Inc., Palo Alto, CA (US)

(72) Inventors: Koji TANABE, Palo Alto, CA (US); Yutaka YASUI, Palo Alto, CA (US)

(73) Assignee: I Peace, Inc., Palo Alto, CA (US)

(21) Appl. No.: 18/792,073

(22) Filed: Aug. 1, 2024

Related U.S. Application Data
(60) Provisional application No. 63/517,262, filed on Aug. 2, 2023.

(51) Int. Cl. C12N 5/0783; A61K 35/17

(52) U.S. Cl. CPC C12N 5/0636; A61K 35/17

(57) ABSTRACT

According to the present disclosure, there is provided a method of producing an immune cell that responds to a cancer-associated antigen, the method including stimulating an immune cell with an antigen contained in a pluripotent stem cell. In addition, according to the present disclosure, there is provided a method of producing an immune cell that responds to a cancer-associated antigen, the method including stimulating an immune cell with at least part of a pluripotent stem cell. In addition, according to the present disclosure, there is provided a method of amplifying response of an immune cell to a cancer-associated antigen, the method including stimulating an immune cell with an antigen contained in a pluripotent stem cell. In addition, according to the present disclosure, there is provided a method of amplifying response of an immune cell to a cancer-associated antigen, the method including stimulating an immune cell with a pluripotent stem cell.

### BACKGROUND

### Field

The present invention relates to a cell technique, and relates to a method of producing an immune cell that responds to a cancer-associated antigen.

### Description of Related Art

Immunotherapies such as cancer vaccine therapy, dendritic cell therapy, and activated T cell therapy are used as cancer treatment methods. However, many of these therapies target single tumor-associated antigens, and even specific antigen epitopes. However, cancer cells are cells that are highly susceptible to gene mutations, and gene expression patterns in cancer tissues are highly diverse. Therefore, it is considered that, in immunotherapies targeting a single tumor antigen or an antigen epitope, no immune response is induced in cancer cells with antigen mutations or decreased expression, and sufficient treatment effects are not obtained.

### CITATION LIST

### Non Patent Literature

[NPL 1] Max Schnurr et al., “Tumor Cell Lysate-pulsed Human Dendritic Cells Induce a T-Cell Response against Pancreatic Carcinoma Cells: an in Vitro Model for the Assessment of Tumor Vaccines,” CANCER RESEARCH 61, 6445-6450 Sep. 1, 2001.

### SUMMARY

One object of the present invention is to provide a method of producing an immune cell that responds to a cancer-associated antigen.

[1] A method of producing an immune cell that responds to a cancer-associated antigen according to an embodiment includes stimulating an immune cell with an antigen contained in a pluripotent stem cell. The antigen contained in the pluripotent stem cell may be at least any of an undifferentiation-associated antigen, an immortalization-associated antigen, and a cell proliferation-associated antigen. The antigen may be at least any of a cancer-testis antigen and a carcinoembryonic antigen. The immune cell and the pluripotent stem cell may be derived from the same subject. The immune cell and the pluripotent stem cell may be derived from the same human. The method may be performed in vivo or in vitro.

[2] In the method of producing the immune cell that responds to the cancer-associated antigen according to the embodiment described in [1], the immune cell may be included in a mononuclear cell. The mononuclear cell and the pluripotent stem cell may be derived from the same subject. The mononuclear cell and the pluripotent stem cell may be derived from the same human.

[3] In the method of producing the immune cell that responds to the cancer-associated antigen according to the embodiment described in [1] or [2], the immune cell may be at least one selected from the group consisting of a T cell, a B cell, a memory B cell, a plasma cell, an NK cell, an NKT cell, a dendritic cell, a monocyte, and a macrophage.

[4] In the method of producing the immune cell that responds to the cancer-associated antigen according to the embodiment described in [3], the T cell may be at least one selected from the group consisting of a helper T cell, a cytotoxic T cell, a regulatory T cell, an αβT cell, and a γδT cell.

[5] In the method of producing the immune cell that responds to the cancer-associated antigen according to the embodiment described in any of [1] to [4], the immune cell may be derived from at least one selected from the group consisting of peripheral blood, cord blood, bone marrow, lymphatic fluid, and tissue fluid.

[6] In the method of producing the immune cell that responds to the cancer-associated antigen according to the embodiment described in any of [1] to [5], the pluripotent stem cell may be an iPS cell.

The method of producing the immune cell that responds to the cancer-associated antigen according to the embodiment described in any of [1] to [6] may further include lysing a pluripotent stem cell to obtain the antigen.

[8] In the method of producing the immune cell that responds to the cancer-associated antigen according to the embodiment described in any of [1] to [7], the antigen may be a lysate of a pluripotent stem cell.

[9] In the method of producing the immune cell that responds to the cancer-associated antigen according to the embodiment described in any of [1] to [8], the stimulated immune cell may respond to a plurality of types of cancer-associated antigens.

[10] In the method of producing the immune cell that responds to the cancer-associated antigen according to the embodiment described in any of [1] to [9], the stimulated immune cell may be at least any of CD69 positive, CD107 positive, and IFN-γ positive.

[11] A method of producing the immune cell that responds to the cancer-associated antigen according to an embodiment, includes stimulating an immune cell with at least part of a pluripotent stem cell. The immune cell and the pluripotent stem cell may be derived from the same subject. The immune cell and the pluripotent stem cell may be derived from the same human. The method may be performed in vivo or in vitro.

[12] In the method of producing the immune cell that responds to the cancer-associated antigen according to the embodiment described in [11], the immune cell may be included in a mononuclear cell. The mononuclear cell and the pluripotent stem cell may be derived from the same subject. The mononuclear cell and the pluripotent stem cell may be derived from the same human.

[13] In the method of producing the immune cell that responds to the cancer-associated antigen according to the embodiment described in [11] or [12], the immune cell may be at least one selected from the group consisting of a T cell, a B cell, a memory B cell, a plasma cell, an NK cell, an NKT cell, a dendritic cell, a monocyte, and a macrophage.

[14] In the method of producing the immune cell that responds to the cancer-associated antigen according to

the embodiment described in [13], the T cell may be at least one selected from the group consisting of a helper T cell, a cytotoxic T cell, a regulatory T cell, an αβT cell, and a γδT cell.

[15] In the method of producing the immune cell that responds to the cancer-associated antigen according to the embodiment described in nay of [11] to [14], the immune cell may be derived from at least one selected from the group consisting of peripheral blood, cord blood, bone marrow, lymphatic fluid, and tissue fluid.

[16] In the method of producing the immune cell that responds to the cancer-associated antigen according to the embodiment described in any of [11] to [15], the pluripotent stem cell may be an iPS cell.

[17] In the method of producing the immune cell that responds to the cancer-associated antigen according to the embodiment described in any of [11] to [16], at least part of the pluripotent stem cell may be a lysate of the pluripotent stem cell.

[18] In the method of producing the immune cell that responds to the cancer-associated antigen according to the embodiment described in any of [11] to [17], the stimulated immune cell may respond to a plurality of types of cancer-associated antigens.

[19] In the method of producing the immune cell that responds to the cancer-associated antigen according to the embodiment described in any of [11] to [18], the stimulated immune cell may be at least any of CD69 positive, CD107 positive, and IFN-γ positive.

[20] A method of amplifying a response of an immune cell to a cancer-associated antigen according to an embodiment, includes stimulating an immune cell with an antigen contained in a pluripotent stem cell. The antigen contained in the pluripotent stem cell may be at least any of an undifferentiation-associated antigen, an immortalization-associated antigen, and a cell proliferation-associated antigen. The antigen may be at least any of a cancer-testis antigen and a carcinoembryonic antigen. The immune cell and the pluripotent stem cell may be derived from the same subject. The immune cell and the pluripotent stem cell may be derived from the same human. The method may be performed in vivo or in vitro.

[21] In the method of amplifying the response of the immune cell to the cancer-associated antigen according to the embodiment described in [20], the immune cell may be included in a mononuclear cell. The mononuclear cell and the pluripotent stem cell may be derived from the same subject. The mononuclear cell and the pluripotent stem cell may be derived from the same human.

[22] In the method of amplifying the response of the immune cell to the cancer-associated antigen according to the embodiment described in [20] or [21], the immune cell may be at least one selected from the group consisting of a T cell, a B cell, a memory B cell, a plasma cell, an NK cell, an NKT cell, a dendritic cell, a monocyte, and a macrophage.

[23] In the method of amplifying the response of the immune cell to the cancer-associated antigen according to the embodiment described in [22], the T cell may be at least one selected from the group consisting of a helper T cell, a cytotoxic T cell, a regulatory T cell, an αβT cell, and a γδT cell.

[24] In the method of amplifying the response of the immune cell to the cancer-associated antigen according to the embodiment described in nay of [20] to [23], the immune cell may be derived from at least one selected from the group consisting of peripheral blood, cord blood, bone marrow, lymphatic fluid, and tissue fluid.

[25] In the method of amplifying the response of the immune cell to the cancer-associated antigen according to the embodiment described in any of [20] to [24], the pluripotent stem cell may be an iPS cell.

[26] The method of amplifying the response of the immune cell to the cancer-associated antigen according to the embodiment described in any of [20] to [25], may further include lysing a pluripotent stem cell to obtain the antigen.

[27] In the method of amplifying the response of immune cell to the cancer-associated antigen according to the embodiment described in any of [20] to [26], the antigen may be a lysate of the pluripotent stem cell.

[28] In the method of amplifying the response of the immune cell to the cancer-associated antigen according to the embodiment described in any of [20] to [27], the stimulated immune cell may respond to a plurality of types of cancer-associated antigens.

[29] In the method of amplifying the response of the immune cell to the cancer-associated antigen according to the embodiment described in any of [20] to [28], the stimulated immune cell may be at least any of CD69 positive, CD107 positive, and IFN-Y positive.

[30] A method of amplifying a response of an immune cell to a cancer-associated antigen according to an embodiment, includes stimulating an immune cell with at least part of a pluripotent stem cell. The immune cell and the pluripotent stem cell may be derived from the same subject. The immune cell and the pluripotent stem cell may be derived from the same human. The method may be performed in vivo or in vitro.

[31] In the method of amplifying the response of the immune cell to the cancer-associated antigen according to the embodiment described in [30], the immune cell may be included in a mononuclear cell. The mononuclear cell and the pluripotent stem cell may be derived from the same subject. The mononuclear cell and the pluripotent stem cell may be derived from the same human.

[32] In the method of amplifying the response of the immune cell to the cancer-associated antigen according to the embodiment described in [30] or [31], the immune cells may be at least one selected from the group consisting of a T cell, a B cell, a memory B cell, a plasma cell, an NK cells, an NKT cells, a dendritic cell, a monocyte, and a macrophage.

[33] In the method of amplifying the response of the immune cell to the cancer-associated antigen according to the embodiment described in [32], the T cell may be at least one selected from the group consisting of a helper T cell, a cytotoxic T cell, a regulatory T cell, an αβT cell, and a γδT cell.

[34] In the method of amplifying the response of the immune cell to the cancer-associated antigen according to the embodiment described in any of [30] to [33], the immune cell may be derived from at least one selected from the group consisting of peripheral blood, cord blood, bone marrow, lymphatic fluid, and tissue fluid.

[35] In the method of amplifying the response of the immune cell to the cancer-associated antigen according to the embodiment described in any of [30] to [34], the pluripotent stem cell may be an iPS cell.

[36] In the method of amplifying the response of the immune cell to the cancer-associated antigen according to the embodiment described in any of to [35], at least part of the pluripotent stem cell may be a lysate of the pluripotent stem cell.

[37] In the method of amplifying the response of the immune cell to the cancer-associated antigen according to the embodiment described in any of [30] to [36], the stimulated immune cell may respond to a plurality of types of cancer-associated antigens.

[38] In the method of amplifying the response of the immune cell to the cancer-associated antigen according to the embodiment described in any of [30] to [37], the stimulated immune cell may be at least any of CD69 positive, CD107 positive, and IFN-γ positive.

[39] An Immune cell that responds to a cancer-associated antigen according to an embodiment is an immune cell stimulated by an antigen contained in a pluripotent stem cell. The antigen contained in the pluripotent stem cell may be at least any of an undifferentiation-associated antigen, an immortalization-associated antigen, and a cell proliferation-associated antigen. The antigen may be at least any of a cancer-testis antigen and a carcinoembryonic antigen. The immune cell and the pluripotent stem cell may be derived from the same subject. The immune cell and the pluripotent stem cell may be derived from the same human.

[40] The immune cell according to the embodiment described in [39] may be included in a mononuclear cell. The mononuclear cell and the pluripotent stem cell may be derived from the same subject. The mononuclear cell and the pluripotent stem cell may be derived from the same human.

[41] The immune cell according to the embodiment described in [39] or [40] may be at least one selected from the group consisting of a T cell, a B cell, a memory B cell, a plasma cell, an NK cell, an NKT cell, a dendritic cell, a monocyte, and a macrophage.

[42] In the immune cell according to the embodiment described in [41], the T cell may be at least one selected from the group consisting of a helper T cell, a cytotoxic T cell, a regulatory T cell, an αβT cell, and a γδT cell.

[43] The immune cell according to the embodiment described in any of [39] to [40] may be derived from at least one selected from the group consisting of peripheral blood, cord blood, bone marrow, lymphatic fluid, and tissue fluid.

[44] In the immune cell according to the embodiment described in any of [39] to [43], the pluripotent stem cell may be an iPS cell.

[45] In the immune cell according to the embodiment described in any of [39] to [44], the antigen may be obtained by lysing the pluripotent stem cell.

[46] In the immune cell according to the embodiment described in any of [39] to [45], the antigen may be a lysate of the pluripotent stem cell.

[47] The immune cell according to the embodiment described in any of [39] to [46] may respond to a plurality of types of cancer-associated antigens.

[48] The immune cell according to the embodiment described in any of [39] to [47] may be at least any of CD69 positive, CD107 positive, and IFN-γ positive.

[49] An immune cell that responds to a cancer-associated antigen according to an embodiment is an immune cell stimulated with at least part of a pluripotent stem cell. The immune cell and the pluripotent stem cell may be derived from the same subject. The immune cell and the pluripotent stem cell may be derived from the same human.

[50] The immune cell according to the embodiment described in [49] may be included in a mononuclear cell. The mononuclear cell and the pluripotent stem cell may be derived from the same subject. The mononuclear cell and the pluripotent stem cell may be derived from the same human.

[51] The immune cell according to the embodiment described in [49] or [50] may be at least one selected from the group consisting of a T cell, a B cell, a memory B cell, a plasma cell, an NK cell, an NKT cell, a dendritic cell, a monocyte, and a macrophage.

[52] In the immune cell according to the embodiment described in [51], the T cell may be at least one selected from the group consisting of a helper T cell, a cytotoxic T cell, a regulatory T cell, an αβT cell, and a γδT cell.

[53] The immune cell according to the embodiment described in any of [49] to [50] may be derived from at least one selected from the group consisting of peripheral blood, cord blood, bone marrow, lymphatic fluid, and tissue fluid.

[54] In the immune cell according to the embodiment described in any of [49] to [53], the pluripotent stem cell may be an iPS cell.

[55] In the immune cell according to the embodiment described in any of [49] to [54], at least part of the pluripotent stem cell may be a lysate of the pluripotent stem cell.

[56] The immune cell according to the embodiment described in any of [49] to [55] may respond to a plurality of types of cancer-associated antigens.

[57] The immune cell according to the

embodiment described in any of [49] to [56] may be at least any of CD69 positive, CD107 positive, and IFN-γ positive.

According to the present invention, it is possible to provide a method of producing an immune cell that responds to a cancer-associated antigen.

### BRIEF DESCRIPTION OF DRAWINGS

FIG. 1 shows dot plots of the results of Example 1 obtained by a flow cytometer;

FIG. 2 shows dot plots of the results of Example 1 obtained by a flow cytometer;

FIG. 3 shows dot plots of the results of Example 2 obtained by a flow cytometer; and

FIG. 4 shows dot plots of the results of Example 2 obtained by a flow cytometer;

### DETAILED DESCRIPTION

Embodiments of the present invention will be described below in detail. Here, the following embodiments are examples for embodying the technical ideas of the invention, and the technical ideas of the invention do not specify the combination of constituent members or the like as in the following. The technical ideas of the invention can be variously modified within the scope of the claims.

A method of producing an immune cell that responds to a cancer-associated antigen according to an embodiment includes stimulating an immune cell with an antigen contained in a pluripotent stem cell or at least part of a pluripotent stem cell. The immune cell and the pluripotent stem cell are derived from, for example, the same subject. The immune cell and the pluripotent stem cell are derived from, for example, the same human individual. When the immune cell and the pluripotent stem cell are derived from different humans, the immune cell is primarily stimulated with a non-self antigen and acquires primary responsiveness against the non-self antigen. On the other hand, when the immune cell and the pluripotent stem cell are derived from the same human, the immune cell primarily acquires responsiveness in response to its own cancer-associated antigen.

The immune cell may be derived from a human or a non-human animal. The immune cell includes, for example, a mononuclear cell. Examples of the immune cell include a

T cell, a B cell, a memory B cell, a plasma cell, an NK cell, an NKT cell, a dendritic cell, a monocyte, and a macrophage. Examples of the T cell include a helper T cell, a cytotoxic T cell, a regulatory T cell, an αβT cell, and a γδT cell.

The immune cell is derived from, for example, blood, bone marrow, thymus, lymphatic fluid, cavity fluid, or tissue fluid. Examples of blood include peripheral blood and cord blood, but the present invention is not limited thereto. The blood may be collected from an adult or a minor. During the blood collection, an anticoagulant such as ethylenediaminetetraacetic acid (EDTA), heparin, and a biological product standard blood preservative solution A (ACD-A) may be used. Examples of the cavity fluid include ascites fluid, pleural fluid, and pericardial fluid. Examples of the tissue include a tumor tissue.

Examples of the pluripotent stem cell include an iPS cell and an ES cell. The pluripotent stem cell is an undifferentiated cell that has an ability to differentiate into all types of cells, and expresses various types of undifferentiation-associated antigens that are not expressed in terminally differentiated cells that constitute organisms. In addition, the pluripotent stem cell expresses immortalization-associated antigens and cell proliferation-associated antigens. Here, in the present disclosure, the antigen includes an epitope. A cancer cell and a tumor cell express different antigens from a normal cell, and antigens expressed by the cancer cell and the tumor cell are called tumor-associated antigens. Antigens included in both undifferentiation-associated antigens and tumor-associated antigens are called “cancer-testis antigens” or “carcinoembryonic antigens.” According to the findings by the inventors, since the pluripotent stem cell includes a plurality of types of cancer-associated antigens, by stimulating the immune cell with at least part of the pluripotent stem cell or its lysate, it is possible to simultaneously make the immune cell responsive to the plurality of types of cancer-associated antigens. The cancer-associated antigens include, for example, cancer-specific antigens. The cancer-associated antigen includes, for example, at least one of a cancer-testis antigen, a cancer stem cell antigen, a cancer undifferentiated antigen, a cancer epithelial antigen, a cancer proliferation-associated antigen, a cancer immortalization antigen, a cancer checkpoint inhibition antigen, a cancer invasion antigen, a cancer surface antigen, a cancer apoptosis inhibition antigen, a cancer epigenetic remodeling antigen, a cancer DNA repair antigen, a cancer microRNA antigen, a cancer nucleic acid antigen, a cancer nuclear receptor antigen, a cancer embryonic antigen, and a carcinoembryonic antigen.

The pluripotent stem cell may be derived from a human. Human-derived pluripotent stem cell may be at least any of TRA1-60 positive, TRA1-85 positive, NANOG positive, LIN28 positive, OCT positive, and Human Nuclear Antigen (HNA) positive. In addition, the pluripotent stem cell includes an undifferentiated naive type cell and a prime type cell with advanced differentiation. A mouse pluripotent stem cell is of a naive type, and a human pluripotent stem cell is of a prime type. The pluripotent stem cell according to the present embodiment may be of the prime type. When the naive pluripotent stem cells are cultured, a leukemia inhibitory factor (LIF) is added to a medium. When the prime type pluripotent stem cells are cultured, a basic fibroblast growth factor (bFGF) is added to a medium. The prime type pluripotent stem cell may be at least any of CD24 positive, CD57 positive, CD90 positive, SSEA4 positive, HLA-A positive, HLA-B positive, and HLA-C positive. In addition, the prime type pluripotent stem cell may be at least any of SSEA1negative, Stella negative, and TFCP2L1 negative.

The antigens contained in the pluripotent stem cell or at least part of the pluripotent stem cells may be, for example, a lysate of the pluripotent stem cells obtained by lysing the pluripotent stem cell. The antigens contained in the pluripotent stem cell or at least part of the pluripotent stem cell may be, for example, a crushed product of the pluripotent stem cell obtained by crushing the pluripotent stem cell. A lysate or crushed product of the pluripotent stem cell may contain various types of undifferentiation-associated antigens, immortalization-associated antigens, and cell proliferation-associated antigens. When the immune cell is stimulated with at least any of the plurality of undifferentiation-associated antigens, immortalization-associated antigens, and cell proliferation-associated antigens contained in the pluripotent stem cell, the immune cell that is responsive to the plurality of cancer antigens is activated. Examples of the cancer antigens include cyclin B1, hyaluronan mediated motility receptor, and heat shock protein 60.

The lysate of the pluripotent stem cell is obtained, for example, by repeatedly freezing and thawing the pluripotent stem cell. The density of the pluripotent stem cells in the solution containing the frozen pluripotent stem cells is, for example, 0.5×10⁴ cells/mL or more and 5.0×10⁶ cells/mL or less, 1.0×10⁴ cells/mL or more and 1×10⁶ cells/mL or less, or 1.5×10⁴ cells/mL or more and 5×10⁵ cells/mL or less, but the present invention is not particularly limited. Alternatively, the lysate of the pluripotent stem cell is obtained, for example, by lysing the pluripotent stem cell with a drug. The crushed product of the pluripotent stem cell is obtained by, for example, applying pressure to the pluripotent stem cell to physically crush the pluripotent stem cell or emitting ultrasonic waves to the pluripotent stem cell to crush the pluripotent stem cell.

The lysate of the pluripotent stem cell may be, for example, an extract of the pluripotent stem cell. The extract of the pluripotent stem cell is produced by the following method. The pluripotent stem cells are cultured on a matrigel coat or a laminin coat using a culture solution. When the pluripotent stem cells are 80% or more confluent, the pluripotent stem cells are scraped off from the culture container using a peeling agent, the solution containing the scraped pluripotent stem cells is centrifuged, and the pluripotent stem cells are collected in a tube. Then, for example, cell masses of the pluripotent stem cells are crushed or ground with a pestle such as a pellet pestle, an ultrasonic crushing device, or a wooden pestle, and an extract solution of the pluripotent stem cells containing a paste of the crushed or ground pluripotent stem cells is frozen with a liquid nitrogen.

The freezing temperature is, for example, −80° C., but the present invention is not particularly limited. Freezing may be instant freezing. According to freezing, the pluripotent stem cells are additionally crushed, and proteins contained in the pluripotent stem cells are released into the extract solution. Here, the pluripotent stem cells may be crushed by freezing without crushing or grinding the pluripotent stem cells in advance. Alternatively, the pluripotent stem cells may be crushed by repeatedly performing freezing and thawing without crushing or grinding the pluripotent stem cells in advance. When the extract solution of the pluripotent stem cells is used, the extract solution of the pluripotent stem cells is suspended in the culture solution and incubated at 4° C. overnight, the suspension is centrifuged the next day, and cell debris is removed from the solution. Then, the solution is filtered through a filter, and the solution that has passed through the filter is used as the extract solution of the pluripotent stem cells.

When the immune cells are stimulated, the

antigens contained in the pluripotent stem cell or at least part of the pluripotent stem cell may be added to a medium in which the immune cells are cultured. The concentration of the lysate of the pluripotent stem cell in the solution containing the lysate of the pluripotent stem cell is, for example, 1 mass % or more and 50 mass % or less, 5 mass % or more and 40 mass % or less, or 10 mass % or more and 30 mass % or less, but the present invention is not particularly limited. The immune cell may be cultured in a medium in which the antigens contained in the pluripotent stem cell or at least part of the pluripotent stem cell are added for 1 day or longer, 3 days or longer, 7 days or longer, or 14 days or longer. Examples of the media in which the immune cells are cultured include an RPMI1640 medium, a minimal essential medium (α-MEM), a Dulbecco's Modified Eagle Medium (DMEM), and an F12 medium, but the present invention is not limited thereto.

When the immune cells are stimulated, a cytokine may be brought into contact with the immune cells, and proliferation of the immune cells may be promoted. Examples of the cytokine include an interleukin. Examples of the interleukin include IL-2, IL-4, IL-7, IL-9, IL-15, IL-18, IL-21, and IL-33. The cytokines may be added to a medium in which the immune cells are cultured. The immune cells may be cultured in a medium in which an anti-cytokine is added for 1 day or longer, 3 days or longer, 7 days or longer, or 14 days or longer.

The immune cells stimulated with the antigens contained in the pluripotent stem cell or at least part of the pluripotent stem cell may respond to, for example, a plurality of types of cancer-associated antigens.

Therefore, even if cancer antigens mutate or their expression levels change in cancer tissues, an appropriate immune effect is maintained compared to immune cells that respond to single cancer-associated antigen. In addition, the stimulated immune cells may be at least any of CD69 positive, CD107 positive, and IFN-γ positive.

### EXAMPLE 1

iPS cells were cultured, and when the colony size exceeded 2 mm or reached 80% confluence, the culture solution was aspirated from the wells, and the iPS cells were washed with PBS(-). PBS(-) was removed from the wells, and TrypLE SELECT (ThermoFisher) was put into the wells. The wells were left in an incubator at 5% CO₂ and 37° C. for 5 minutes. Then, the TrypLE SELECT was removed from the wells, and the iPS cells were washed with PBS(-). PBS(-) was removed from the wells, a medium to which an ROCK inhibitor was added was put into the wells, and the iPS cells were scraped off from the wells using a cell scraper.

The number of iPS cells was measured, and a cell suspension containing 2.5×10⁵ iPS cells was put into a 15 mL tube. The tube was centrifuged at 200×g for five minutes, and the supernatant was aspirated. A medium was put into the tube, and the iPS cells were suspended by pipetting.

iPS cells suspended in a medium that did not contain cell freezing solution were put into a freezer at −80° C. for one hour, the cell suspension was frozen and the cells were destroyed. The cell survival rate was 1% or less. Then, the cell suspension was thawed at room temperature, and the cell suspension was mixed using a vortex mixer. The iPS cells were crushed by repeating these freezing and thawing steps a total of four times. Then, the tube was centrifuged at 300×g for 10 minutes, and the supernatant containing the iPS cell lysate was collected and put into a 1.5 mL tube. The supernatant was passed through a 0.22 μm filter to remove cell debris from the supernatant, and was put into another 1.5 mL tube as an iPS cell lysate solution.

An RPMI1640 medium (ThermoFisher) containing 10% of FBS, 1% of GlutaMax, 1% of non-essential amino acid (NEAA), 1% of sodium pyruvate, 55 μmol/L of mercaptoethanol, and 1% of penicillin streptomycin was prepared as an amplification medium.

Mononuclear cells derived from peripheral blood were added to the amplification medium in the test tube. iPS cells and peripheral blood were derived from the same human individual. Next, 20% of the iPS cell lysate solution, 1 μg/mL of anti-CD28 antibodies, and 100 U/mL of IL-2 were added to the amplification medium, culturing of the mononuclear cells in an incubator at 5% CO₂ and 37° C. started, and immune cells that specifically responded to antigens contained in iPS cells were activated, proliferated and amplified in vitro. On the 3rd day after culturing of mononuclear cells started, half of the medium was replaced with a fresh amplification medium. On the 7^{th} day after culturing of mononuclear cells started, the cells were collected, the collected cells were added to a fresh amplification medium, the iPS cell lysate solution, anti-CD28 antibodies, and IL-2 were added to the amplification medium. On the 10^{th} day after culturing of mononuclear cells started, half of the medium was replaced with a fresh amplification medium. On the 14^{th} day after culturing of mononuclear cells started, the cells were collected.

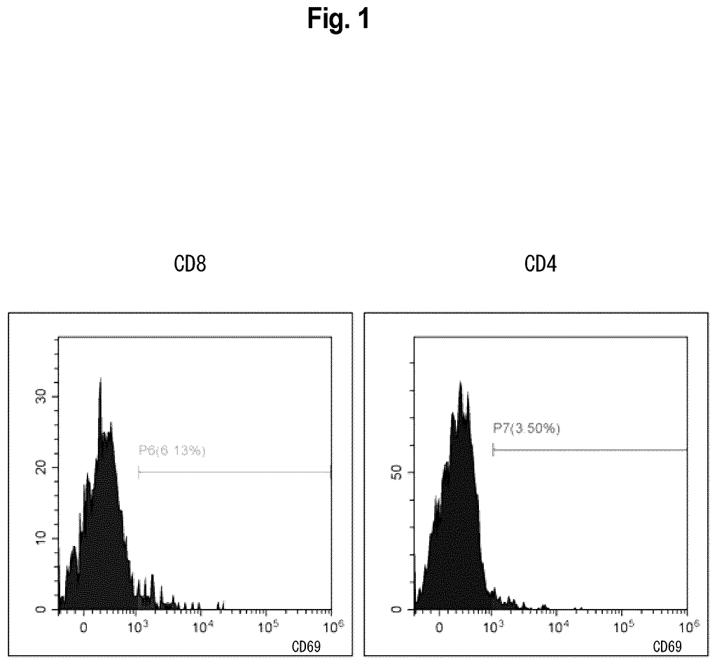

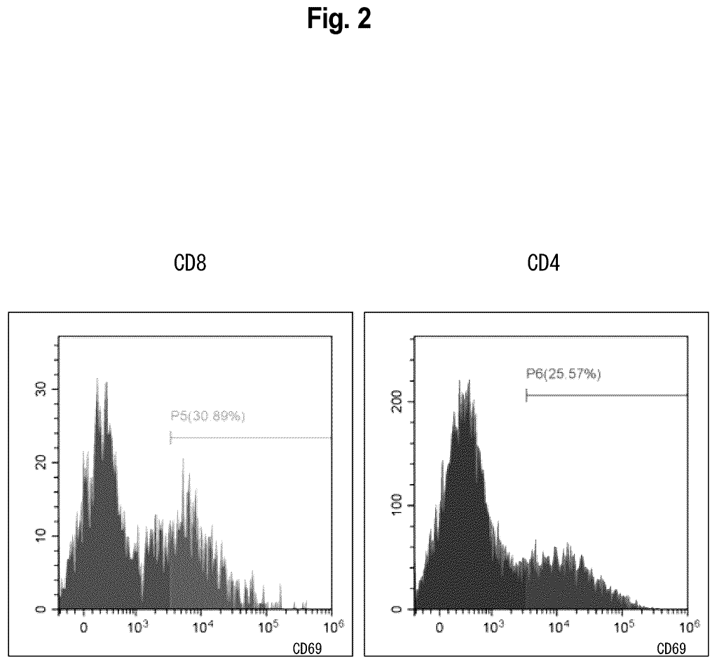

Mononuclear cells prior to being cultured in the amplification medium containing the iPS cell lysate solution were analyzed through flow cytometry. As shown in FIG. 1, neither CD8-positive T cells nor CD4-positive T cells expressed an activation marker CD69. On the other hand, as shown in FIG. 2, in cells derived from mononuclear cells cultured and amplified in an amplification medium containing the iPS cell lysate solution, both CD8-positive T cells and CD4-positive T cells expressed the activation marker CD69, and included amplified and activated cells.

### EXAMPLE 2

By the same method as in Example 1, mononuclear cells cultured in an amplification medium containing an iPS cell lysate solution for 14 days were collected and washed with PBS. As cancer antigen peptides, a cyclin B1 peptide, a hyaluronan mediated motility receptor (HMMR) peptide, and a heat shock protein 60 (HSP60) peptide were prepared, and each of them was added to the amplification medium at a concentration of 1 μmol/L. In addition, as a negative control, amplification media containing no cancer antigen peptide were prepared, the mononuclear cells were added to each amplification medium, and the mononuclear cells were cultured in an incubator at 5% CO2 and 37° C.

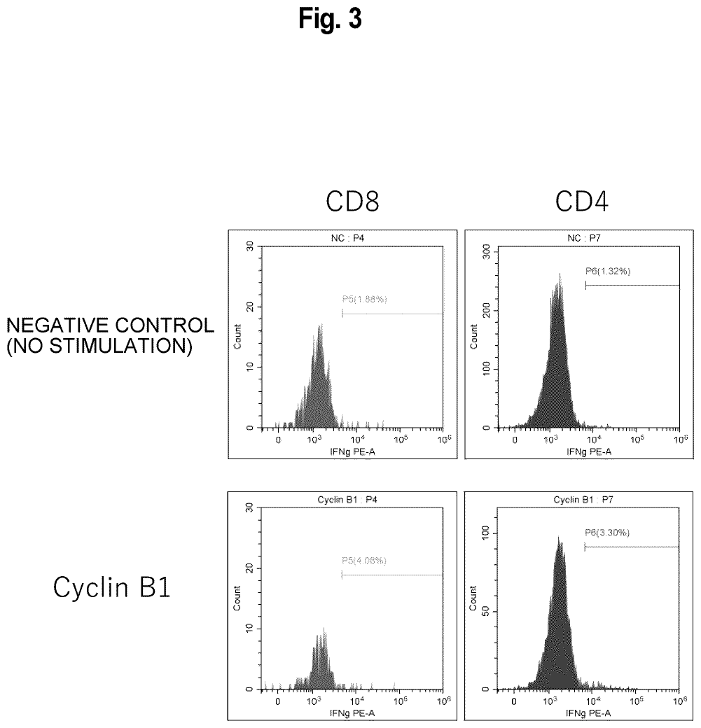

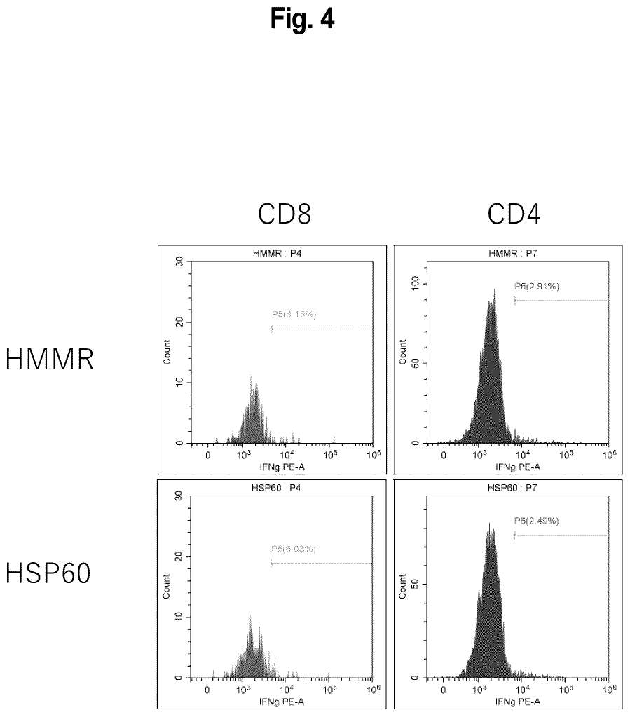

After five hours, the mononuclear cells were collected, the mononuclear cells were stained using an IFN-γ secretion assay detection kit (Miltenyi Biotec, 130-054-202), anti-CD4 antibodies, and anti-CD8 antibodies, and the mononuclear cells were analyzed using a flow cytometer. As shown in FIG. 3 and FIG. 4, in both CD8-positive T cells and CD4-positive T cells stimulated with the cyclin B1 peptide, the HMMR peptide, and the HSP60 peptide, release of an activation marker IFN-γ was observed. In both CD8-positive T cells and CD4-positive T cells in the negative control, less activation marker IFN-γ release was observed.

1. A method of producing an immune cell that responds to a cancer-associated antigen, the method comprising
stimulating an immune cell with an antigen contained in a pluripotent stem cell.

2. The method according to claim 1,
wherein the immune cell is included in a mononuclear cell.

3. The method according to claim 1,
wherein the immune cell is at least one selected from the group consisting of a T cell, a B cell, a memory B cell, a plasma cell, an NK cell, an NKT cell, a dendritic cell, a monocyte, and a macrophage.

4. The method according to claim 1, further comprising
lysing the pluripotent stem cell to obtain the antigen.

5. The method according to claim 1,
wherein the antigen is a lysate of a pluripotent stem cell.

6. The method according to claim 1,
wherein the stimulated immune cell responds to a plurality of types of cancer-associated antigens.

7. The method according to claim 1,
wherein the stimulated immune cell is at least one of CD69 positive, CD107 positive, and IFN-γ positive.

8. A method of producing an immune cell that responds to a cancer-associated antigen, the method comprising
stimulating an immune cell with at least part of a pluripotent stem cell.

9. The method according to claim 8,
wherein the immune cell is included in a mononuclear cell.

10. The method according to claim 8,
wherein at least part of the pluripotent stem cell is a lysate of a pluripotent stem cell.

11. The method according to claim 8,
wherein the stimulated immune cell responds to a plurality of types of cancer-associated antigens.

12. The method according to claim 8,
wherein the stimulated immune cell is at least one of CD69 positive, CD107 positive, and IFN-γ positive.

13. A method of amplifying a response of an immune cell to a cancer-associated antigen, the method comprising
stimulating an immune cell with an antigen contained in a pluripotent stem cell.

14. A method of amplifying a response of an immune cell to a cancer-associated antigen, the method comprising
stimulating an immune cell with at least part of a pluripotent stem cell.

15. A pharmaceutical composition for cancer treatment, comprising
an immune cell stimulated with an antigen contained in a pluripotent stem cell.

16. A pharmaceutical composition for cancer treatment, comprising
an immune cell stimulated with at least part of a pluripotent stem cell.

17. An immune cell that responds to a cancer-associated antigen stimulated by an antigen contained in a pluripotent stem cell.

18. An immune cell that respond to a cancer-associated antigen stimulated by at least part of a pluripotent stem cell.
